(19) United States
(12) Patent Application Publication
(10) Pub. No.: US 2013/0178935 A1
(43) Pub. Date: Jul. 11, 2013

(54) POLARIZED COMPONENT OCULAR DEVICES

(71) Applicants: Lara Henry, Goleta, CA (US); Jensen Buck, Goleta, CA (US); Anand Doraiswamy, Goleta, CA (US)

(72) Inventors: Lara Henry, Goleta, CA (US); Jensen Buck, Goleta, CA (US); Anand Doraiswamy, Goleta, CA (US)

(21) Appl. No.: 13/347,996

(22) Filed: Jan. 11, 2012

(51) Int. Cl. A61F 2/16

(52) U.S. Cl. 623/6.17

(57) ABSTRACT

Described are ocular devices such as intraocular lens preferably made of an organic polymeric material and an ultraviolet light absorber coated with a polarizing polymer component adjacent the edge thereof. A typical polarizing component is polyvinyl alcohol doped with iodine (PVA/I₂). The ocular device with an added polarizing component is capable of absorbing light parallel to the axis of the polymer chain, while transmitting light that is perpendicular to the axis of the polymer chains. The polarizing component will reduce polarized light radiating off of horizontal surfaces; therefore, the film will improve vision through the device by reducing glare from light sources.

### BACKGROUND OF THE INVENTION

1. Field of Invention

This invention relates to ocular devices such as intraocular lenses wherein the lens is an anterior chamber, iris slip, or posterior chamber intraocular lens. Currently, commercially available intraocular lenses provide increased absorbance of UV light from unfiltered light sources with UV absorbing substrates in the polymer blend of the lens. Blue light blocking technology also exists in the market place to improve the vision of patients with intraocular lenses implants. Patients with implanted intraocular lenses often see what is known as edge glare. Edge glare refers to light rays that focus on the retina and create high contrast in the vision of images seen by the patient. Lens edge glare generally falls into the categories of discomfort glare and disability glare. Discomfort glare encourages a patient to look away from an image or environment to avoid glare to avoid discomfort whereas disability glare inhibits visibility with bright light sources.

2. Description of Prior Art

To reduce edge glare, various edge designs have been discovered and implemented in the intraocular lens implant market. In particular, edge designs include frosting, rounding, and sharpening of the edge of the lens.

The edge design selected must also eliminate or decrease the amount of posterior capsule opacification (PCO) that requires some intraocular lens implant patients to have additional surgery. PCO, sometimes called “secondary cataract,” is the most common long-term complication of modern cataract surgery. The problem develops when the posterior portion of the capsular bag—the structure which encloses the eye's natural lense—begins to get cloudy. The posterior portion of this bag is left intact during cataract surgery to prevent leakage of vitreous from behind the capsule, which could cause a detached retina. PCO can occur months or years after cataract surgery, unlike most other cataract surgery complications that then to occur during or shortly after the procedure.

Within 2 to 5 years after surgery, PCO causes decreased visual acuity in 20 percent to 40 percent of eyes that have had cataract surgery. The problem is age-dependent and occurs more frequently in young patients. The rate of PCO among children who have surgery to remove congenital cataracts ranges from 44 percent to 100 percent.

Opacification of the posterior capsule appears to be influenced by lens epithelial cells that are left behind in the eye during cataract removal. The cells multiply, migrate across the posterior lens capsule and undergo changes that cause fibrous or pearl-type opacities in the capsule. The exact mechanism in not completely understood, but it appears to be an aberrant wound healing response to cataract surgery.

Currently, the only treatment for PCO is a type of laser surgery called Nd:YAG laser capsulotomy. In this procedure, a laser is used to create a central opening in the cloudy posterior capsule to restore sight. The procedure is quick and easy, but possible complications of laser capsulotomy include retinal detachment, damage to the intraocular lens (IOC), increase in intraocular pressure and other problems. “Posterior capsular opacification: A problem reduced but not yet eradicated.” Archives of Ophthalmology, April 2009, incorporated by reference herein.

In many clinical studies, the effect of frosting on the lens edge only reduces the glare to a partial ring. That design does not prevent PCO. Further clinical trials indicate that the round edge intraocular lenses do not reduce the amount of PCO experienced by implant patients. The sharp edge design of an intraocular lens indicates reduced PCO formation.

### SUMMARY OF THE INVENTION

Briefly, in one aspect, the present invention is an improvement to the intraocular lens art. In particular, this invention involves modification of the edge of an ocular device or an annular ring surface or area adjacent to the edge of an ocular device to reduce glare induced by unfiltered light sources. The modification is to apply a coating, layer, polymer, or copolymer of a polarizing component or polarizing element, adjacent to the edge (including upon or on the edge) of an ocular device. A preferred ocular device of this invention is an intraocular lens (IOL). Generally the polarizing component is applied in an annular ring leaving the lens center un-coated.

Applying a polarizing component to the lens periphery or lens edge according to this invention produces better vision by reducing contrast between unfiltered light sources during the day, such as the sun, and shiny objects, such as the surface of a car. Applications of this invention also tends to eliminate or reduce glare induced by e.g., the head lights of oncoming cars or street lights, during the night without decreasing the concentration of light waves that pass through the optic.

It is theorized that development of PCO may also be inhibited or stopped by application of this invention.

The application of a polarizing component as a coating to a lens edge or lens body, according to this invention, will not increase the overall volume of the lens. Nor will this component interfere with the geometry of the lens edges.

It is understood that the present invention involves the application of a polarizing component, generally a light polarizing polymer, adjacent to the edge of an ocular device with the intention of reducing the common forms of glare. The terminology “adjacent to the edge” of an ocular device is intended to include: (a) any actual lens edge, i.e., a surface disposed between the anterior and posterior surface of a lens and being thereby defined by that surface (as opposed to a sharp edge) the edge being a circular band having its center coaxial with the axis of the lens; (b) the annular surface immediately proximate the lens edge on either or both of the anterior or posterior lens surfaces; or (the disjunctive being intentionally chosen) (c), both (a) and (b). This definition means generally that the center of the lens has no polarizing component e.g., a coating thereon.

Specifically, a polarizing component of this invention will cover no more than about 50% of the lens surfaces adjacent the lens edge; preferably no more than about 30% of the lens surface adjacent the lens edge and most preferably no more than about 20% of the lens surface adjacent the lens edge

### DESCRIPTION OF THE DRAWINGS

The following drawings which are a part of the invention serve to explain the properties of the invention.

FIG. 1A is a front view of a 6.0 mm 20.0 D one piece intraocular lens wherein the darker area on the optic represents the maximum extent of the polarizing component deposition. Numeral 1 is the radius of the polarizing component and, in this embodiment, is limited to less than or equal to about 2 mm radially inward from the edge of the lens.

FIG. 1B is a side view of a 6.0 mm 20.0 D one piece intraocular lens wherein the polarizing component covers the side of the optic, but not the haptic. Numeral 30 indicates the edge of the lens covered by the polarizing film. As is shown, edge 40 is not sharp but has an anterior and/or posterior surface on which the coating is disposed.

FIG. 2A is a front view of a 7.0 mm 20.0 D one piece intraocular lens wherein the darker area 50 on the optic represents the maximum extent of the polarizing component deposition. Numeral 3 is the radius of the polarizing component and is limited to less than or equal to about 3.5 mm radially disposed inward from edge 40.

FIG. 2B is a side view of a 7.0 mm 20.0 D one piece intraocular lens wherein the polarizing component covers the side of the optic, but not the haptic. Numeral 60 indicates the edge of the lens covered by the polarizing component.

### DETAILED DESCRIPTION OF THE INVENTION

The present invention utilizes a structured deployment of a polarizing component or coating adjacent to or on the edge of a lens body, (sometimes referred to as the “optic” of a lens) generally without coating any of the lens support structure or fixation means e.g. its haptics. A preferred lens body comprises a optically acceptable polymeric material, the polarizer component comprising poly(vinyl) alcohol (PVA) doped with iodine. PVA-Iodine complexes are extensively discussed in the Miyasaka reference noted below. The polarizer may be a single layer, multiple layers or multiple layers of polarizer having the same axes of polarization. The polarizer may also be a mixture, or a copolymer, preferably doped with I₂.

A polymeric lens body or optic of this invention may comprise any optically acceptable polymer to which a polarizer coating of this invention can be permanently bonded. The lens body may be a rigid polymer or preferably a flexible polymeric material e.g., to make a foldable completed lens. Hydrophobic and hydrophilic acrylic materials containing acrylate and methacrylate functionalities are preferred. See, also U.S. Pat. No. 4,916,197 to Vacik et al and. U.S. Pat. No. 5,359,021 to Weinschenk, III.

Miyasaka et al in their review article noted below discuss numerous techniques and methods for formation of polyvinyl alcohol (PVA)/iodine complexes which are the preferred coating material for use in the present invention. At pages 91-96 Miyasaka et al. describe numerous routes for preparation of PVA/iodine complexes each of those preparative routes being specifically incorporated by reference herein. For purposes of this invention generally, and specifically for purposes of PVA/Iodine film polarizers, a PVA film may be first applied to a lens body with polarizer film preparation completed by soaking the PVA film in a solution, e.g., an aqueous solution, of an iodine salt. Further a polarizer film may be directly applied to the lens body from a film solution, e.g., a PVA/Iodine film solution. It is also possible to utilize an iodine/vinyl alcohol monomer solution or mixture which is applied to the lens optic or lens body, the vinyl alcohol monomer then being polymerized to create the polarizer film, in place, e.g., by heating or exposure to UV radiation. Numerous other approaches to aggressively and permanently adhering or bonding a polarizing film to a lens body according to this invention will be suggested to one skilled in this art by the present disclosure.

Polyvinylene-Iodine alone or in combination with PVA is another polarizer coating composition or material to be used in the present invention.

Multiple layers of polarizing components or coatings, including multiple layers of polarizing components of different chemical compositions also are contemplated.

Block copolymers having polarizing characteristics are also contemplated by this invention.

It is noted, in on aspect, that the present invention involves the placement of a polarizing film adjacent to the edge of an ocular device for glare reduction. Numerous techniques are known, and may be used to place, the polarizer film in its intended location on a lens body or optic body. For example dip coating, spin coating, plasma etch and chemical vapor deposition are techniques that could be used. Masking techniques, e.g., masking of the center of the lens body or optic, the mask being removed after placement of the polarizing element or coating to create an uncoated center, is another approach that can be used.

Lenses of this invention, particularly intraocular lenses, comprise a lens body optic with optional supporting haptics. The optic has a central axis, an anterior face, an opposing posterior face and a peripheral edge or edge surface between the anterior and posterior faces. The optic is adapted for placement in the capsular bag of the eye and to direct light toward the retina.

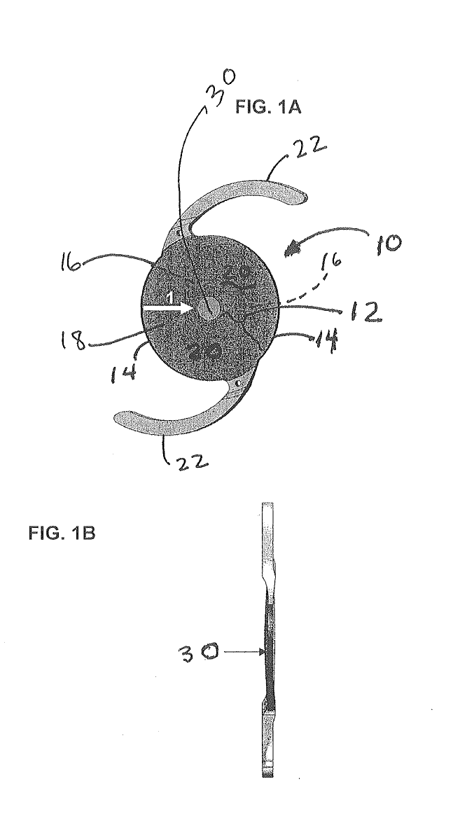

Thus there is shown in FIG. 1A a front view of a 6.0 mm, 20.0 D intraocular lens 10. Lens 10 comprises a lens body or optic 20 having two sides, 16, 18 (reference to the back side of lens 10 being made by the dashed lead line) and an edge 14. Edge 14 does not come to a peak to define a sharp edge but is a surface which couples sides 16, 18. Either of sides 16, 18 may define the anterior or posterior lens surface depending upon lens orientation relative to the patient's eye. Lens haptics 22 are shown. The darker area adjacent the optic perimeter (designated by bracket 12) shows the extent of the polarizing component deposition on lens body 20. Numeral 1 is the designation for an arrow illustrating the radius of the polarizing component coating area, or region. In the embodiment shown, the edge-disposed polarizing coat or layer 12 extends inwardly from the edge 14 of lens 10 a radial distance of, at most, about 2 mm. Uncoated central zone or region 30 also is shown. Also in this embodiment, lens haptics 22 have an optional coating or layer or a polarizing material or coating, reducing glare produced by the haptics.

FIG. 1B depicts a device similar to that of FIG. 1A with the exception that no polarizing component is coated upon the haptic. Also of significance is the presence of a polarizing coat on edge 40. Edge 40 has an actual radial surface in the shape of a band as viewed from the side of the lens, the center thereof being the optical center of the lens body. The center of the lens edge would also generally be collinear with the optical axis of the lens (not shown).

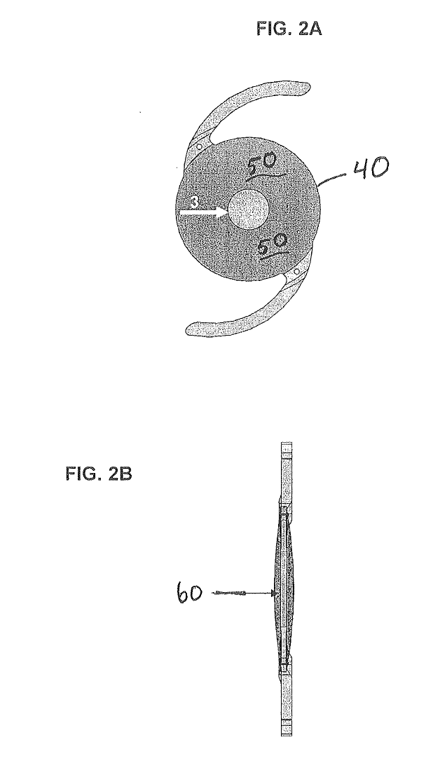

FIGS. 2A and 2B show front and side views of a slightly larger (7 .mm) single piece intraocular lens the structures being analogous to those of the lens shown in FIGS. 1A and 1B.

It will be appreciated that the preferred ophthalmic application or device for application of the present invention is an intraocular lens (IOL). One skilled in the IOL art will appreciate that the material selected to produce the IOL must be selected in view of its intended use. For example, the material selected, e.g., for the optic must be optically-acceptable, flexible if, e.g., a foldable lens is chosen, and biocompatible so as not to cause patient immune or other response. “Implantable” would be another characterization of an appropriate optic polymer material.

The following US Patent Documents and Other Publications are specifically incorporated by reference into this application.

### U.S. PATENT DOCUMENTS

| | | |
|---|---|---|
| 5,044,743 | August 1991 | Ting |
| 4,755,182 | July 1988 | Holmes |
| 6,884,262 | April 2005 | Brady et al. |
| 5,666,223 | September 1997 | Bennett et al. |
| 6,224,945 | May 2001 | Calderara |
| 7,811,402 B2 | October 2010 | Nimura |
| 7,717,556 | May 2010 | Walker |
| 6,874,888 | April 2005 | Dudai |

### OTHER PUBLICATIONS

Han, Man, et al., “Preparation of Syndiotacticity-Rich High Molecular Weight Poly(vinyl alcohol)/Iodine Polarizing Film with High Water Resistance”, Wiley InterScience: Vol. 115, pp. 917-922 (2010).
Miyasaka, K, “PVA-Iodine Complexes: Formation, Structure, and Properties”, Advances in Polymer Science Vol. 108, pp. 91-129 (1993).
Villain, et al., “Flexible Intraocular Lens Made of Hydrophilic Acrylic Material,” Publication No. EP 0946219

The above description and attached figures are intended to be illustrative and not limiting of the above invention which is defined in the following claims.

What is claimed is as follows:

1. An intraocular lens comprising:
(a) an organic, optically-acceptable polymer optic body the optic body having an edge and including thereon;
(b) a polarizing component comprising one or more incident light polarizing polymers the polarizing component being coated on to the optic body adjacent to its edge.

2. An intraocular lens according to claim 1 wherein the lens is a foldable lens.

3. An intraocular lens according to claim 1 wherein the polarizing component is a d-block copolymer.

4. An intraocular lens according to claim 1 wherein the polarizing component comprises one or more layers of a polymer.

5. An intraocular lens according to claim 1 wherein the polarizing component is composed of poly (vinyl alcohol) doped with iodine.

6. An intraocular lens according to claim 1 wherein the polarizing component comprises polyvinylene.

7. An intraocular lens according to claim 1 wherein the polarizing component comprises one or more layers of polyvinylene and poly (vinyl alcohol) doped with iodine.

8. An intraocular lens according to claim 1 wherein the polarizing component is composed of one or more layers of a copolymer containing polyvinylene and poly (vinyl alcohol) doped with iodine.

9. An intraocular lens according to claim 1 wherein the polarizing component is an amorphous polymer.

10. An intraocular lens according to claim 1 wherein the polarizing component covers the edge of the intraocular lens.

11. An intraocular lens according to claim 1 wherein the polarizing component covers only the edge of the intraocular lens.

12. An intraocular lens according to claim 1 wherein the optically acceptable polymer material further comprises an ultraviolet light-absorbing material.

13. An intraocular lens comprising:
(a) an organic, optically-acceptable, chronically implantable polymer optic body, the optic body having an edge and anterior and posterior surfaces, the optic body including thereon;
(b) a polarizing component comprising incident-light polarizing polymer, the polarizing component being coated on the optic body adjacent its edge.

14. An intraocular lens according to claim 13 wherein the polarizing component covers less than 50% of the posterior surface of the optic body.

15. An intraocular lens according to claim 13 wherein the polarizing component covers portions of only the anterior surface of the optic body adjacent its edge.

16. An intraocular lens according to claim 13 wherein the polarizing component covers the posterior and anterior surface of the optic.

17. An intraocular lens according to claim 13 wherein the polarizing component is coated upon on any flat plane between the posterior and anterior surfaces of the optic body.

18. An intraocular lens according to claim 13 wherein the polarizing component is on an angled plane that is symmetric about the left and right hand side of the lens and between the posterior and anterior surfaces of the optic body.

19. An intraocular lens according to claim 13 wherein the polarizing component is one or more layers of polarizing polymer film.

20. An intraocular lens according to claim 13 wherein the polarizing component covers a radius on the 6.00 mm lens up to 2.00 mm from the edge of the optic body.

21. An intraocular lens according to claim 13 wherein the polarizing component covers a radius on the 7.00 mm lens up to 4.50 mm from the edge of the optic body.

22. An intraocular lens according to claim 13 wherein the polarizing component comprises a radius on the 6.00 mm lens up to 2.00 mm from the edge of the optic body.

23. An intraocular lens according to claim 13 wherein the polarizing component comprises a radius on the 7.00 mm lens up to 4.50 mm from the edge of the optic body.

24. An intraocular lens according to claim 13 wherein the polarizing component is etched onto the surface of the optic body at any radius up to 2.00 mm from the edge of the optic body for the 6.00 mm lens.

25. An intraocular lens according to claim 13 wherein the polarizing component is etched onto the surface of the optic body at any radius up to 4.50 mm from the edge of the optic body for the 7.00 mm lens.

26. An intraocular lens according to claim 13 wherein the polarizing component is etched onto the surface of the optic body at any radius up to 2.00 mm from the edge of the optic body for the 6.00 mm lens and has a radius less than or equal to 2.00 mm.

27. An intraocular lens according to claim 13 wherein the polarizing component is etched onto the surface of the optic body at any radius up to 4.50 mm from the edge of the optic body for the 7.00 mm lens and has a radius less than or equal to 4.50 mm.

28. An ophthalmic device comprising:
(a) an optically-acceptable polymer optic body the optic body having an edge and including thereon;
(b) a polarizing component comprising one or more incident light polarizing polymers the polarizing component being coated on to the optic body adjacent to its edge.
